(19) United States
(12) Patent Application Publication
(10) Pub. No.: US 2020/0381102 A1
(43) Pub. Date: Dec. 3, 2020

(54) Intelligent Robot-Based Rehabilitation Training Method For Patient With Dementia

(71) Applicant: Dongguan BaiHe Biological Technology Co., Ltd., Dongguan (CN)

(72) Inventors: Yong He, Dongguan (CN); Kejie Lv, Dongguan (CN); Hang Zhang, Dongguan (CN)

(73) Assignee: Dongguan BaiHe Biological Technology Co., Ltd., Dongguan (CN)

(21) Appl. No.: 16/891,330

(22) Filed: Jun. 3, 2020

(30) Foreign Application Priority Data
Jun. 3, 2019 (CN) 201910475724.4

(51) Int. Cl. G16H 20/70; G16H 40/67; G06F 3/01; G06F 3/0484; G02B 27/01

(52) U.S. Cl. CPC G16H 20/70; G16H 40/67; G06F 3/011; G02B 2027/0178; G02B 27/0172; G02B 2027/0174; G06F 3/0484

(57) ABSTRACT

An intelligent robot-based rehabilitation training method for patients with dementia includes: a. collecting and storing physical condition data and personal hobby data of patients with dementia, so that a robot formulates a personalized rehabilitation training program, where the program includes a visual rehabilitation training method and a memory rehabilitation training method; b. in the visual rehabilitation training method, displaying image, animation or video information on a display device, and performing human-computer interaction with the patient to determine the patient's visual recognition level according to a result of the interaction between the patient and the robot, and stimulate and train the patient's visual recognition ability; and c. in the memory rehabilitation training method, displaying a combination of one or more of objects, colors, numbers, smells, pictures, past memories, puzzles, dementia quantification tables, and clock drawings on the display screen, and performing human-computer interaction with the patient to stimulate.

### CROSS REFERENCE TO RELATED APPLICATIONS

This application claims priority from Chinese application number 201910475724.4, filed Jun. 3, 2019, the disclosure of which is hereby incorporated herein by reference.

### TECHNICAL FIELD

The present invention relates to the technical field of robots, and in particular, to a rehabilitation training method for patients with dementia, to minimize the suffering of those patients, reduce the care burden of their family members, reduce the work intensity of nursing staff, and help the rehabilitation of the patients.

### BACKGROUND

Normal people show their cognitive processes such as sensation, perception, memory, thinking, imagination, and language in their daily behaviors. The normal people's brain nerves, body immune function, and intestinal flora structure are in the normal range. However, patients with dementia have different manifestations, such as memory impairment, aphasia, apraxia, loss of recognition, impaired visuospatial skills, executive dysfunction, and personality and behavior changes. Patients with dementia show memory loss and decreased judgment ability at the early stage of the disease. They are unable to analyze, think, and judge, difficult to deal with complex problems and locate their geographic locations, have social difficulties, poor visuospatial skills for complex structures, and few words and phrases, and cannot do shopping or deal with economic affairs independently. Patients with dementia in moderate dementia show severely impaired near and far memory, reduced visuospatial skills for simple structures, time and place disorientation, and severely impaired capacity in handling problems and identifying similarities and differences between things. They cannot do outdoor activities independently, need help in dressing and keeping their personal hygiene and appearance, and cannot calculate, along with various neurological symptoms. Patients with dementia in severe dementia can only rely on caregivers. They suffer severe memory loss, have only a few memories, cannot take care of themselves, and show incontinence, silence, and stiff limbs, where physical examination result shows positive pyramidal signs. They have primitive reflexes such as strong grip, groping, and sucking, and eventually fall into a coma, and usually die from complications such as infection.

In addition, Alzheimer's disease is a progressive degenerative encephalopathy that is considered as irreversible. Currently, there are no specific drugs and treatments, and the development of the disease can only be delayed through human care. The traditional method is the combination of drug treatment and home nursing. However, due to the impaired or severely impaired cognitive ability, memory ability, and judgment ability of patients with dementia, drug treatment and traditional nursing methods have very limited effects. The workload of nursing staff is extremely large but the rehabilitation effect is small.

### SUMMARY

To solve the above problems, the present invention provides an intelligent robot-based rehabilitation training method for patients with dementia, to perform rehabilitation training through human-computer interaction, so as to effectively maintain or improve the cognitive ability of the patients with dementia and prevent their cognitive ability, memory ability, and judgment ability from getting worse. This can minimize the suffering of the patients, reduce the burden of family members and the work intensity of nursing staff, and help the patients to gradually recover.

The intelligent robot-based rehabilitation training method for patients with dementia includes the following steps.

a. Collect and store physical condition data and personal hobby data of a patient with dementia, so that the robot formulates a personalized rehabilitation training program based on the collected data, where the program includes a visual rehabilitation training method and a memory rehabilitation training method, and the visual rehabilitation training method and the memory rehabilitation training method are performed through human-computer interaction between the patient and the robot.

b. In the visual rehabilitation training method, display image, animation or video information on a display device, and perform human-computer interaction with the patient, to determine the patient's visual recognition level according to a result of the interaction between the patient and the robot, and stimulate and train the patient's visual recognition ability.

c. In the memory rehabilitation training method, display a combination of one or more of objects, colors, numbers, smells, pictures, past memories, puzzles, dementia quantification tables, and clock drawings on the display screen, and perform human-computer interaction with the patient to stimulate and train the patient's memory recovery ability, and determine the patient's memory recovery status.

In step b, in the visual rehabilitation training method, the robot controls the display device to display the image, animation or video information, and the patient wears LED glasses or VR glasses to watch. The robot asks a question based on the displayed content, and the patient answers in voice. When the answer is correct, points are accumulated and a next screen is displayed. When the answer is incorrect, points are deducted and a next screen is displayed. This training can be performed regularly or irregularly.

In the visual rehabilitation training method, the robot controls the display device to display 3D hologram images, and asks a question based on the displayed content, and the patient answers in voice. When the answer is correct, points are accumulated and a next screen is displayed. When the answer is incorrect, points are deducted and a next screen is displayed. This training can be performed regularly or irregularly.

In step b, the display device is one of a 3D advertising fan, a smartwatch, an automatic cooking robot, an automatic dryer, an automatic pill box, and a cleaning robot, and is provided with a display screen. The display device is connected to and communicates with the robot in a wireless manner.

In step c, the memory rehabilitation training method is a combination of one or more of a clock drawing training method, a dementia quantification table test and training method, a spatial ability training method, a recall training method, a digital calculation training method, a color confusion training method, and an object recognition training method.

In the clock drawing training method, the robot displays a clock pattern on the display screen, and the patient selects a color to fill in the clock pattern. This training helps the patient restore the cognition of numbers and improve spatial cognition abilities.

In the dementia quantification table test and training method, the robot displays a dementia quantification table on the display screen. The table contains common sense, memory, calculation, and recognition memory quantification indicators for scoring and evaluation of the patient. The robot displays common sense, memory, and calculation questions on the display screen, and the patient answers in voice. When the answer is correct, points are accumulated and a next screen is displayed. When the answer is incorrect, points are deducted and a next screen is displayed. The robot calculates the total score based on the points of the correct answers and forms recognition memory quantification indicators. This training can be performed regularly or irregularly.

In the spatial ability training method, the robot displays a jigsaw puzzle interface on the display screen, which includes a complete picture and several pieces divided from the picture. The patient combines the pieces into the complete picture. This training helps train the patient's brain thinking ability and improve the imagination ability and spatial cognition ability.

In the recall training method, the robot displays pictures, audios, videos or 3D holographic content of the patient's familiar people or things on the display screen, and asks questions based on the displayed content. The patient answers in voice. When the answer is correct, points are accumulated and a next screen is displayed. When the answer is incorrect, points are deducted and a next screen is displayed. This training helps the patient to recognize the familiar people or things, and helps determine the patient's historical memory and training levels and stimulate the patient's memory. This training can be performed regularly or irregularly.

In the digital calculation training method, the robot displays a graphical digital calculation interface on the display screen, and the patient calculates and fills in a calculation result. If the result is correct, points are accumulated and a next screen is displayed. If the result is incorrect, points are deducted and a next screen is displayed. This training helps improve the patient's brain activity, ensure the normal interaction between the patient and the robot, and train brain regions corresponding to hands and language.

In the color confusion training method, the robot displays several groups of confusing colored objects on the display screen, and the patient fills color in different object and finds the correct answer from the confusing colored objects. This training helps strengthen the ability to memorize objects of different colors.

In the object recognition training method, the robot displays a variety of objects in different positions on the display screen, and asks the patient to find matched object pairs. The patient finds the matched objects quickly. This training helps the patient to recall the objects as much as possible, to strengthen the patient's ability to recall objects, and stimulate and train the patient's memory recovery ability.

The method further includes a robot nursing service for patients with dementia, including question-and-answer language communication between a patient and a robot. The robot opens audiovisual programs and searches for encyclopedia knowledge and peripheral services according to the patient's voice instructions. The language communication and information query with the robot helps stimulate the recovery of the patient's recognition ability, memory ability, and understanding ability.

The present invention effectively solves the problems that patients with dementia rely merely on medication, require a lot of human care, but lack necessary physical rehabilitation training methods. The method of the present invention is specially designed for patients with dementia based on the thinking characteristics of the human brain. In this method, a robot performs physical rehabilitation training for patients through human-computer interaction. The present invention provides various training methods, including visual training, memory training, and robot nursing, designed for patients with dementia with impaired or severely impaired cognitive ability, memory ability, and judgment ability. The visual training content provided by the robot helps determine the patient's visual recognition level, and stimulate and train the visual recognition ability of the patient. Human-computer interaction with the aid of objects, colors, numbers, smells, pictures, past memories, puzzles, dementia quantification tables, and clock drawings can stimulate and train the memory recovery ability of the patient. Nursing services such as intimate conversations, encyclopedia knowledge and information query, and peripheral services can partially replace manual nursing, which can effectively reduce the burden of family members and the work intensity of nursing staff. The present invention may be widely used in communities, families, nursing homes, and rehabilitation centers to improve the health of patients with dementia through physical rehabilitation training.

### BRIEF DESCRIPTION OF DRAWINGS

FIG. 1 is a flowchart of a method according to the present invention.

FIG. 2 is a structural block diagram of a robot.

FIG. 3 is a flowchart of a visual rehabilitation training method according to the present invention.

FIG. 4A-4G is a flowchart of a memory rehabilitation training method, where FIG. 4A is a flowchart of a clock drawing training method, FIG. 4B is a flowchart of a dementia quantification table test and training method, FIG. 4C is a flowchart of a spatial ability training method, FIG. 4D is a flowchart of a recall training method, FIG. 4E is a flowchart of a digital calculation training method, FIG. 4F is a flowchart of a color confusion training method, and FIG. 4G is a flowchart of an object recognition training method.

FIG. 5 is a flowchart of a nursing service method according to the present invention.

FIG. 6 is a schematic diagram of interaction between a patient, a robot and a display device according to the present invention.

### DETAILED DESCRIPTION

The following examples are further explanations and supplements to the present invention and do not constitute any limitation to the present invention.

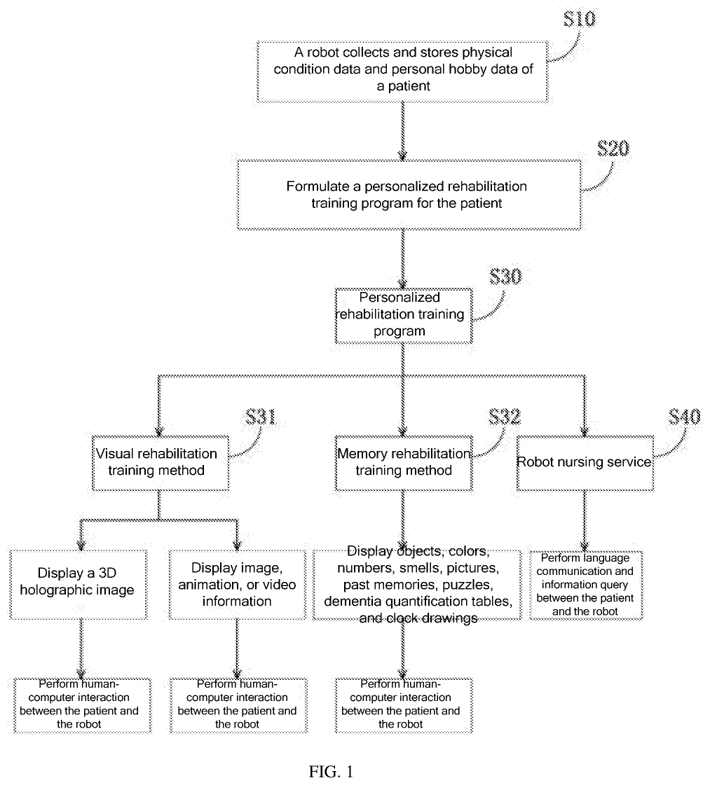

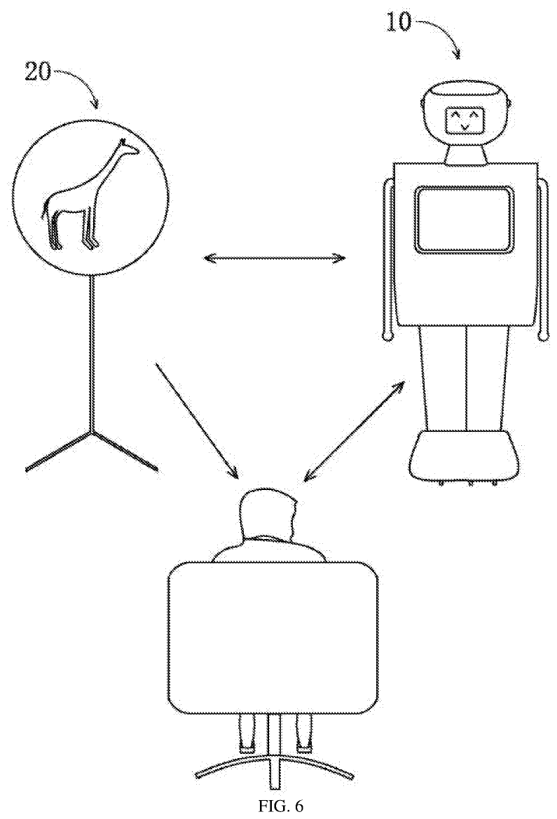

As shown in FIG. 1 and FIG. 6, an intelligent robot-based rehabilitation training method for patients with dementia according to the present invention includes collecting and storing personalized data of patients with dementia, where a rehabilitation training program includes a visual rehabilitation training method, a memory rehabilitation training method, and a robot nursing service.

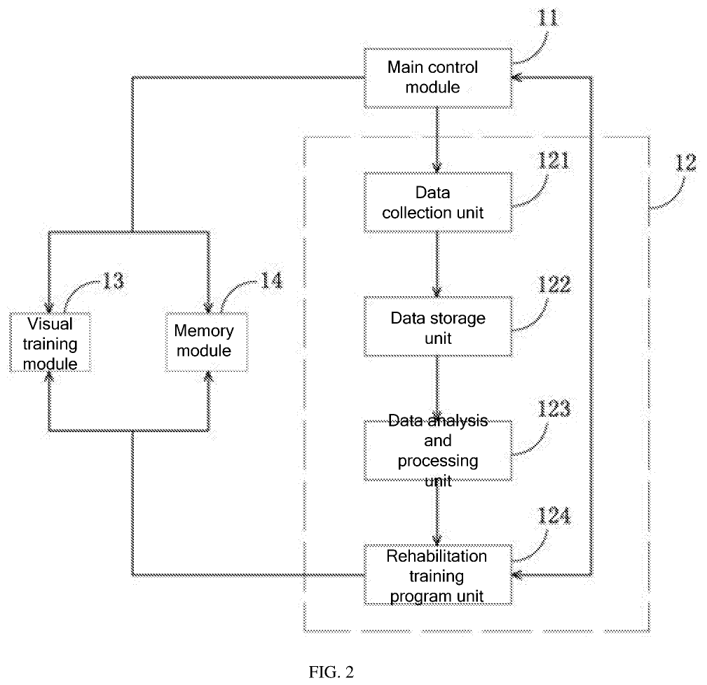

S10: Collect and store physical condition data and personal hobby data of patients with dementia, where physical condition data and personal hobby data of different patients with dementia are collected and stored, and data collection and analysis are performed by a robot 10. As shown in FIG. 2, the robot 10 includes a main control module 11 and a personalized service module 12. The personalized service module 12 is a functional module loaded with software programs, and includes a data collection unit 121, a data storage unit 122, a data analysis and processing unit 123, and a rehabilitation training program unit 124. The data collection unit 121 is configured to collect the physical condition data of patients output through hospital, telephone, or video consultations, or surveys with relatives and friends of the patients. The data collection unit 121 also collects the personal hobby data of the patients, such as singing or listening to music, and traveling. The personalized service module 12 is electrically connected to the main control module 11 and the data storage unit 122. The data collected by the data collection unit 121 is stored in the data storage unit 122 as medical records. The data analysis and processing unit 123 is electrically connected to the data storage unit 122 and the rehabilitation training program unit 124, and is configured to analyze the physical data of different patients stored in the data storage unit 122, and provide personalized solutions for different patients.

S20: The data analysis and processing unit 123 sends the processed data to the rehabilitation training program unit 124. The rehabilitation training program unit 124 formulates a personalized rehabilitation training program suitable for each patient based on the solution provided by the data analysis and processing unit 123, and sends the program to the main control module 11. The rehabilitation training program includes content, steps, time, and effect evaluation of the rehabilitation training.

S30: The personalized rehabilitation training program includes a visual rehabilitation training method and a memory rehabilitation training method, as shown in FIG. 6. The visual rehabilitation training method and the memory rehabilitation training method are performed through human-computer interaction between the patient and the robot. The two training methods can be performed separately or crossly.

S31: Visual Rehabilitation Training Method

As shown in FIG. 2, the robot has a visual training module 13, which is an execution module of the personalized service module 12. The robot performs visual training on patients through the visual training module. In the visual rehabilitation training method, the robot displays image, animation or video information on a display device, and performs human-computer interaction with the patient to determine the patient's visual recognition level according to a result of the interaction between the patient and the robot, and stimulate and train the patient's visual recognition ability. The visual training may be a combination of one or more of LED glasses training, VR glasses training, and 3D holographic image training. In the LED glasses training and VR glasses training, the patient wears LED glasses and VR glasses, and watches the training content through the LED glasses and VR glasses. The LED glasses training and VR glasses training further include a human-computer interaction control program and a display device 20 that cooperates with the LED glasses and VR glasses. The display device 20 may be one of a 3D advertising fan, a smartwatch, an automatic cooking robot, an automatic dryer, an automatic pill box, and a cleaning robot, and stores the image, animation, or video information for visual training. The 3D holographic images for the 3D holographic image training are stored and displayed on the display device 20.

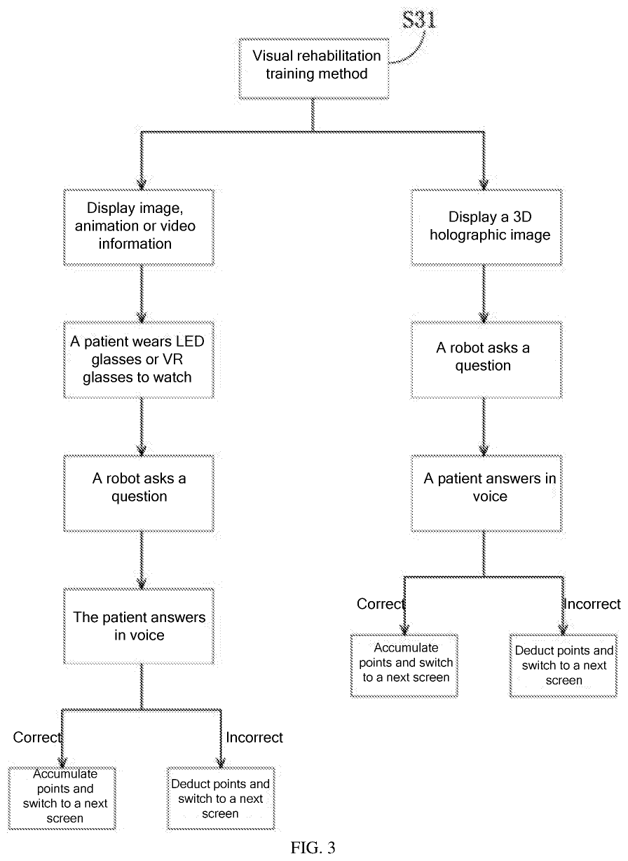

As shown in FIG. 3, in one example of the visual rehabilitation training method, the visual training module 13 of the robot 10 controls the display device 20 to display the image, animation, or video information, such as videos of the loved ones, childhood or youth photos of the patient, and once-familiar music and songs, and the patient wears LED glasses or VR glasses to watch. For example, when a bicycle is displayed on the display screen, the robot asks, “What do you see?”, and the patient answers in voice. If the patient answers “bicycle”, the answer is correct, and the robot automatically accumulates points and switches to a next screen. If the answer is incorrect, the robot automatically deducts points and switches to a next screen. This training can be performed regularly or irregularly.

As shown in FIG. 3, in another example of the visual rehabilitation training method, the visual training module 13 of the robot 10 controls the display device 20 to display a 3D holographic image. In this example, the display device is a 3D advertising fan. For example, when a giraffe is displayed on a display screen of the 3D advertising fan, the robot asks: “What do you see?”, and the patient answers in voice. If the patient answers “giraffe”, the answer is correct, and the robot automatically accumulates points and switches to a next screen. If the answer is incorrect, the robot automatically deducts points and switches to a next screen. This training can be performed regularly or irregularly.

Through this visual rehabilitation training method, realistic visual stimulation and inductive rehabilitation training can be performed on patients.

S32: Memory Rehabilitation Training Method

As shown in FIG. 2, the robot 10 has a memory module 14, which is an execution module of the personalized service module 12. The memory module 14 includes a combination of one or more of a clock drawing training method, a dementia quantification table test and training method, a spatial ability training method, a recall training method, a digital calculation training method, a color confusion training method, and an object recognition training method. The robot 10 displays a combination of one or more of objects, colors, numbers, smells, pictures, past memories, puzzles, dementia quantification tables, and clock drawings on the display screen through the memory module 14, and interacts with the patient to stimulate and train the patient's memory recovery ability, and determine the patient's memory recovery status.

S321: Clock Drawing Training Method

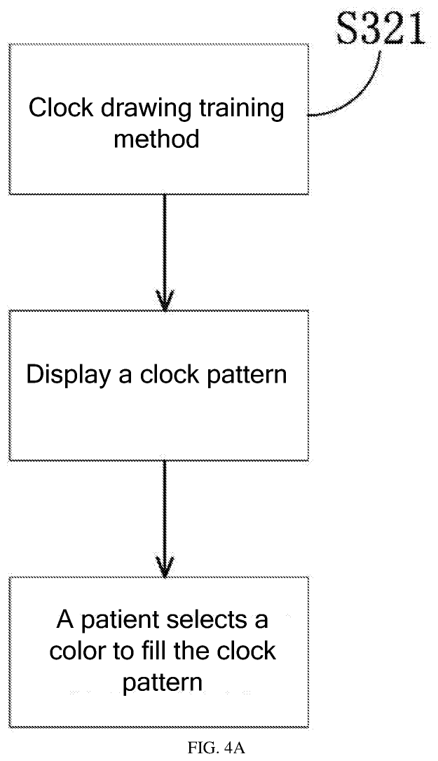

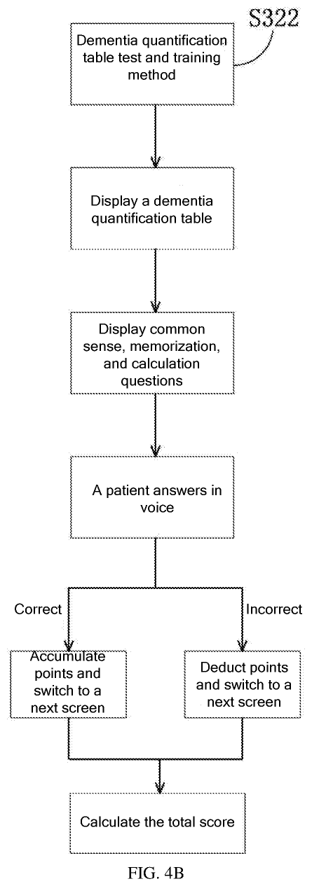

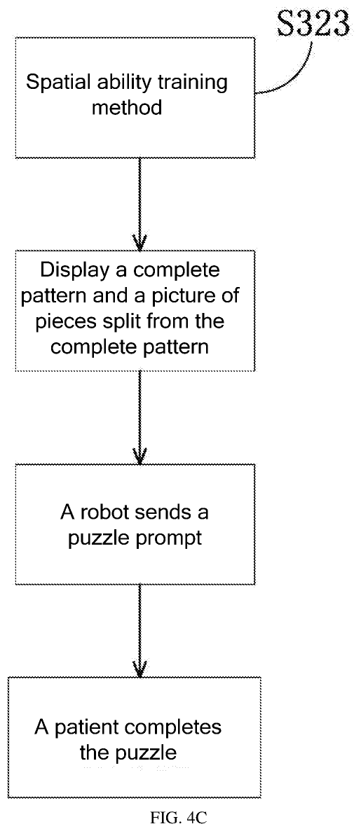

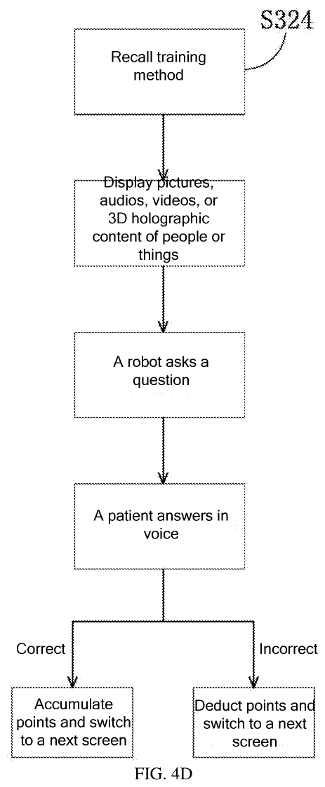

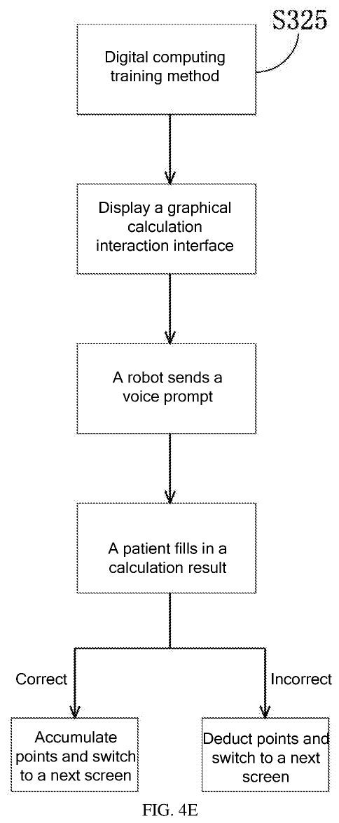

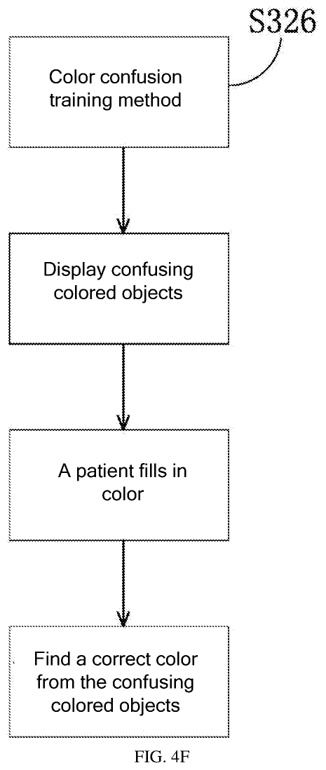

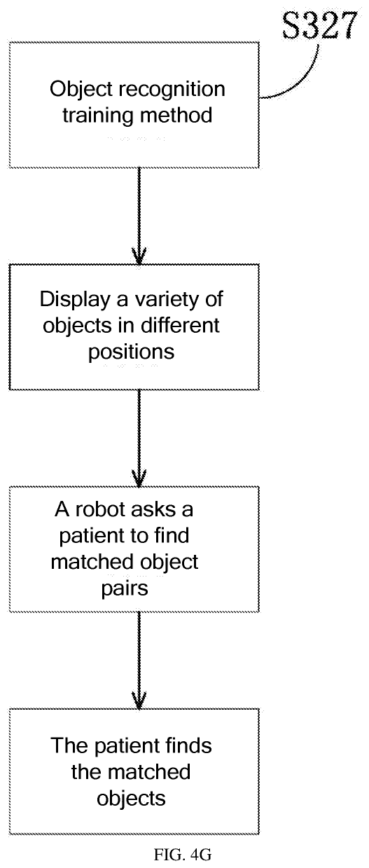

As shown in FIG. 4A, in the clock drawing training method, the robot 10 displays an analog clock pattern on the display screen, and the patient selects a color to fill in the clock pattern. This training helps the patient restore the cognition of numbers and improve spatial cognition abilities.

S322: Dementia Quantification Table Test and Training Method

As shown in FIG. 4B, in the dementia quantification table test and training method, the robot 10 displays a dementia quantification table on the display screen. The table includes common sense, memory, calculation, and recognition memory quantization indicators. During the training, the robot 10 displays common sense, memorization, and calculation questions on the display screen, and asks questions. The patient answers each question in voice, and the robot gives scores and evaluation for each question. When the answer is correct, the robot 10 accumulates points and switches to a next screen. When the answer is incorrect, the robot 10 deducts points and switches to a next screen. The robot calculates the total score based on the points of the correct answers and forms recognition memory quantification indicators. For example, the robot asks the patient to recite “6-8-2” backwards for recognition memory training, with 5 points accumulated for the correct answer. The robot asks “how old are you” for memory training, with 5 points accumulated for the correct answer (±3 years is correct). The robot asks the patient to make simple arithmetic calculations for calculation training, for example, 100-7, with 4 points accumulated for the correct answer. The robot asks the patient to answer common sense questions for common sense training, with 10 points accumulated for the correct answer. The total score is calculated to obtain a recognition memory quantification indicator. The recognition memory quantification indicator greater than 30.2 indicates normal, that ranging from 30.5 to 22 indicates subnormal, that ranging from 21.5 to 10.5 indicates suspected dementia, and that ranging from 10 to 0 indicates dementia. This training helps determine whether the patient has cognitive impairment. Based on the patient's cognitive situation and the input parameters of different questions, the cognitive distribution in different parts of the patient's brain can be determined, so that more targeted rehabilitation training can be performed to strengthen the patient's memory. This training can be performed regularly or irregularly.

S323: Spatial Ability Training Method

The spatial ability training method is designed for patients with insufficient spatial imagination or patients with cognitive impairment. As shown in FIG. 4C, in this example, two pictures are displayed on the display screen of the robot 10: One is a complete flower pattern, and the other is a picture of pieces split from the flower pattern. After the robot gives a puzzle instruction, the patient tries to complete the puzzle. This training helps train the brain's thinking ability and improve the imagination ability and spatial cognition ability. Obviously, the difficulty of the spatial ability training can be determined according to the patient's condition, and different patterns can be used for each training.

S324: Recall Training Method

The recall training method is a kind of picture-centric multimedia-aided training designed for improving the patient's long-term memory or for the situation that the patient is unable to remember related people and things. During the recall training, the robot displays pictures, audios, videos, or 3D holographic content of the patient's familiar people or things on the display screen. This training helps the patient to recognize the familiar people or things, and helps determine the patient's historical memory and training levels and stimulate the patient's memory. As shown in FIG. 4D, in this example, a family photo is displayed on the display screen of the robot 60, and several options of grandson, son, daughter-in-law, and yourself are displayed below the photo. The robot asks, “Who is the first person, counted from left to right?” If the patient answers “my son”, the answer is correct, and the robot automatically accumulates points and switches to a next screen. If the answer is incorrect, the robot automatically deducts points and switches to a next screen. This training can wake up the historical memory stored in the hippocampus of the patient's brain, gradually enabling the patient to recall the previous people and things and make memory state feedback on familiar people and things. The patient's answers can accurately reflect the training situation of the patient. This training can be performed regularly or irregularly.

S325: Digital Computing Training Method

The digital computing training method is designed for patients who have lost digital computing functions. During digital computing training, the robot displays a graphical calculation interaction interface on the display screen, and the patient performs the calculation. As shown in FIG. 4E, in this example, a graphical calculation formula “05+02=?” is displayed on the display screen. The robot sends a voice prompt to the patient: “Please calculate”, and the patient fills in the calculation result. If the answer is correct, the robot automatically accumulates points and switches to a next screen. If the answer is incorrect, the robot automatically deducts points and switches to a next screen. The digital computing training can improve the patient's brain activity, ensure normal human-computer interaction, train the brain regions corresponding to hands and language, and restore the patient's computing ability.

S326: Color Confusion Training Method

The color confusion training method is designed for patients who cannot distinguish between colors and have lost memory of color. During the color confusion training, the robot displays confusing colored objects on the display screen, and the patient finds the correct answer based on the colors of different objects. This training helps strengthen the ability to memorize objects of different colors. As shown in FIG. 4F, the robot displays text in a certain color and three options on the display screen, where the three options confuse different semantics in different colors. The patient is asked to find out the correct color expressed by the text with a certain color. When the robot sends a voice prompt to the patient: “Please select the correct color of the text”, the patient finds the correct answer from the confusing colored texts. This training can strengthen the ability to memorize objects with different colors and select the correct color, so that the patient can restore the memory of color as much as possible.

S327: Object Recognition Training Method

The object recognition training method is designed for patients who have lost memory of different objects. As shown in FIG. 4G, the robot displays confusing colored objects on the display screen, such as fruits, animals, articles, celestial bodies, and mountains. The patient is required to accurately find two identical objects in the shielded grids to strengthen the memory of objects with different colors. As shown in the figure, the robot 60 displays several groups of fruits on the display screen, with the same fruits randomly distributed in different grids. When the robot sends a voice prompt to the patient: “Please find two identical fruits”. If the patient finds the same fruits, lines of the grids disappear. If the patient does not find the same fruits, the grid lines continue to exist. This training can be performed regularly or irregularly. This training helps the patient to recall objects as much as possible.

S40: Robot Nursing Service

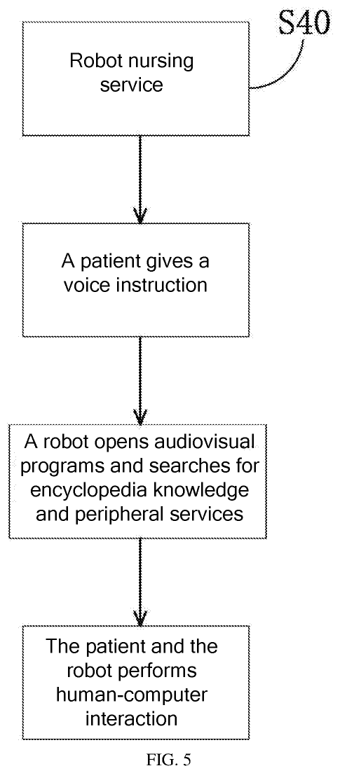

The present invention further includes a robot nursing service for patients with dementia. The service includes question-and-answer language communication between a patient and a robot. The robot opens audiovisual programs and searches for encyclopedia knowledge and peripheral services according to the patient's voice instructions. In the present invention, the robot is provided with an intelligent voice communication platform, which can undertake some of the nursing tasks of the nursing staff. As shown in FIG. 5, in this nursing service, the patient can communicate with the robot through the intelligent voice communication platform, such as daily chat with the robot and intelligent companionship chat in a question-and-answer manner The patient can also ask the robot for stocks, map navigation, little jokes, common sense, music, songs, and knowledge. Conversation and information query can stimulate recovery of the patient's recognition ability, memory ability, and understanding ability. This also effectively reduces the burden of nursing staff.

To sum up, the present invention provides a rehabilitation training method for patients with dementia. This method is based on the thinking characteristics of the human brain. The robot performs physical rehabilitation training for patients in a human-computer interaction mode, and provides personalized rehabilitation training programs for patients with dementia based on their specific conditions. The present invention uses comprehensive intelligent rehabilitation training, including visual training, memory training, and robot nursing service, to effectively maintain or improve the cognitive ability of patients with dementia and prevent their cognitive ability, memory ability, and judgment ability from getting worse. This can minimize the suffering of the patients, reduce the burden of family members and the work intensity of nursing staff, and help the patients to gradually recover.

Although the present invention has been disclosed through the above examples, the protection scope of the present invention is not limited thereto. The modifications and replacements made without departing from the concept of the present invention shall fall into the scope of the claims of the present invention.

1. An intelligent robot-based rehabilitation training method for patients with dementia, comprising:
a. collecting and storing physical condition data and personal hobby data of patients with dementia, so that a robot formulates a personalized rehabilitation training program based on the collected data, wherein the program comprises a visual rehabilitation training method and a memory rehabilitation training method, and the visual rehabilitation training method and the memory rehabilitation training method are performed through human-computer interaction between the patient and the robot;
b. in the visual rehabilitation training method, displaying image, animation or video information on a display device, and performing human-computer interaction with the patient to determine the patient's visual recognition level according to a result of the interaction between the patient and the robot, and stimulate and train the patient's visual recognition ability; and
c. in the memory rehabilitation training method, displaying a combination of one or more of objects, colors, numbers, smells, pictures, past memories, puzzles, dementia quantification tables, and clock drawings on the display screen, and performing human-computer interaction with the patient to stimulate and train the patient's memory recovery ability, and determine the patient's memory recovery status.

2. The intelligent robot-based rehabilitation training method for patients with dementia according to claim 1, wherein in step b, in the visual rehabilitation training method, the robot controls the display device to display the image, animation or video information, and the patient wears LED glasses or VR glasses to watch; the robot asks questions based on the displayed content, and the patient answers in voice; when the answer is correct, points are accumulated and a next screen is displayed; when the answer is incorrect, points are deducted and a next screen is displayed; and this training can be performed regularly or irregularly.

3. The intelligent robot-based rehabilitation training method for patients with dementia according to claim 1, wherein in the visual rehabilitation training method, the robot controls the display device to display 3D hologram images, and asks questions based on the displayed content, and the patient answers in voice; when the answer is correct, points are accumulated and a next screen is displayed; when the answer is incorrect, points are deducted and a next screen is displayed; and this training can be performed regularly or irregularly.

4. The intelligent robot-based rehabilitation training method for patients with dementia according to claim 2, wherein in step b, the display device is one of a 3D advertising fan, a smartwatch, an automatic cooking robot, an automatic dryer, an automatic pill box, and a cleaning robot, and is provided with a display screen; and the display device is connected to and communicates with the robot in a wireless manner.

5. The intelligent robot-based rehabilitation training method for patients with dementia according to claim 4, wherein in step c, the memory rehabilitation training method is a combination of one or more of a clock drawing training method, a dementia quantification table test and training method, a spatial ability training method, a recall training method, a digital calculation training method, a color confusion training method, and an object recognition training method.

6. The intelligent robot-based rehabilitation training method for patients with dementia according to claim 5, wherein in the clock drawing training method, the robot displays a clock pattern on the display screen, and the patient selects a color to fill in the clock pattern; and this training helps the patient to restore the cognition of numbers and improve spatial cognition abilities.

7. The intelligent robot-based rehabilitation training method for patients with dementia according to claim 5, wherein in the dementia quantification table test and training method, the robot displays a dementia quantification table on the display screen, wherein the table contains common sense, memory, calculation, and recognition memory quantification indicators for scoring and evaluation of the patient; the robot displays common sense, memory, and calculation questions on the display screen, and the patient answers in voice; when the answer is correct, points are accumulated and a next screen is displayed; when the answer is incorrect, points are deducted and a next screen is displayed; the robot calculates the total score based on the points of the correct answers and forms recognition memory quantification indicators; and this training can be performed regularly or irregularly.

8. The intelligent robot-based rehabilitation training method for patients with dementia according to claim 5, wherein in the spatial ability training method, the robot displays a jigsaw puzzle interface on the display screen, wherein the interface comprises a complete picture and several pieces divided from the picture; the patient combines the pieces into the complete picture; and this training helps train the patient's brain thinking ability and improve the imagination ability and spatial cognition ability.

9. The intelligent robot-based rehabilitation training method for patients with dementia according to claim 5, wherein in the recall training method, the robot displays pictures, audios, videos, or 3D holographic content of the patient's familiar people or things on the display screen, and asks questions based on the displayed content, and the patient answers in voice; when the answer is correct, points are accumulated and a next screen is displayed; when the answer is incorrect, points are deducted and a next screen is displayed; this training helps the patient to recognize the familiar people or things, and helps determine the patient's historical memory and training levels and stimulate the patient's memory; and this training can be performed regularly or irregularly.

10. The intelligent robot-based rehabilitation training method for patients with dementia according to claim 5, wherein in the digital calculation training method, the robot displays a graphical digital calculation interface on the display screen, and the patient calculates and fills in a calculation result; if the result is correct, points are accumulated and a next screen is displayed; if the result is incorrect, points are deducted and a next screen is displayed; this training helps improve the patient's brain activity, ensure the normal interaction between the patient and the robot, and train brain regions corresponding to hands and language.

11. The intelligent robot-based rehabilitation training method for patients with dementia according to claim 5, wherein in the color confusion training method, the robot displays several groups of confusing colored objects on the display screen, and the patient fills color in different object and finds the correct answer from the confusing colored objects; and this training helps strengthen the ability to memorize objects of different colors.

12. The intelligent robot-based rehabilitation training method for patients with dementia according to claim 5, wherein in the object recognition training method, the robot displays a variety of objects in different positions on the display screen, and asks the patient to find matched object pairs; the patient finds the matched objects quickly; and this training helps the patient to recall the objects as much as possible, and helps strengthen the patient's ability to recall objects, and stimulate and train the patient's memory recovery ability.

13. The intelligent robot-based rehabilitation training method for patients with dementia according to claim 1, wherein the method further comprises a robot nursing service for patients with dementia, comprising question-and-answer language communication between the patient and the robot; the robot opens audiovisual programs and searches for encyclopedia knowledge and peripheral services according to the patient's voice instructions; and the language communication and information query with the robot stimulate the recovery of the patient's recognition ability, memory ability, and understanding ability.

14. The intelligent robot-based rehabilitation training method for patients with dementia according to claim 3, wherein in step b, the display device is one of a 3D advertising fan, a smartwatch, an automatic cooking robot, an automatic dryer, an automatic pill box, and a cleaning robot, and is provided with a display screen; and the display device is connected to and communicates with the robot in a wireless manner.

15. The intelligent robot-based rehabilitation training method for patients with dementia according to claim 14, wherein in step c, the memory rehabilitation training method is a combination of one or more of a clock drawing training method, a dementia quantification table test and training method, a spatial ability training method, a recall training method, a digital calculation training method, a color confusion training method, and an object recognition training method.

16. The intelligent robot-based rehabilitation training method for patients with dementia according to claim 15, wherein in the clock drawing training method, the robot displays a clock pattern on the display screen, and the patient selects a color to fill in the clock pattern; and this training helps the patient to restore the cognition of numbers and improve spatial cognition abilities.

17. The intelligent robot-based rehabilitation training method for patients with dementia according to claim 15, wherein in the dementia quantification table test and training method, the robot displays a dementia quantification table on the display screen, wherein the table contains common sense, memory, calculation, and recognition memory quantification indicators for scoring and evaluation of the patient; the robot displays common sense, memory, and calculation questions on the display screen, and the patient answers in voice; when the answer is correct, points are accumulated and a next screen is displayed; when the answer is incorrect, points are deducted and a next screen is displayed; the robot calculates the total score based on the points of the correct answers and forms recognition memory quantification indicators; and this training can be performed regularly or irregularly.

18. The intelligent robot-based rehabilitation training method for patients with dementia according to claim 15, wherein in the spatial ability training method, the robot displays a jigsaw puzzle interface on the display screen, wherein the interface comprises a complete picture and several pieces divided from the picture; the patient combines the pieces into the complete picture; and
this training helps train the patient's brain thinking ability and improve the imagination ability and spatial cognition ability.

19. The intelligent robot-based rehabilitation training method for patients with dementia according to claim 15, wherein in the recall training method, the robot displays pictures, audios, videos, or 3D holographic content of the patient's familiar people or things on the display screen, and asks questions based on the displayed content, and the patient answers in voice; when the answer is correct, points are accumulated and a next screen is displayed; when the answer is incorrect, points are deducted and a next screen is displayed; this training helps the patient to recognize the familiar people or things, and helps determine the patient's historical memory and training levels and stimulate the patient's memory; and this training can be performed regularly or irregularly.

20. The intelligent robot-based rehabilitation training method for patients with dementia according to claim 15, wherein in the digital calculation training method, the robot displays a graphical digital calculation interface on the display screen, and the patient calculates and fills in a calculation result; if the result is correct, points are accumulated and a next screen is displayed; if the result is incorrect, points are deducted and a next screen is displayed; this training helps improve the patient's brain activity, ensure the normal interaction between the patient and the robot, and train brain regions corresponding to hands and language.
